(19) United States
(12) Patent Application Publication
(10) Pub. No.: US 2025/0100979 A1
(43) Pub. Date: Mar. 27, 2025

(54) SYNTHESIS OF A TETRAZOLE DERIVATIVE

(71) Applicants: BRAJA SUNDAR PRADHAN, Hyderabad (IN); NEHA MEHTA, Picatinny Arsenal, NJ (US); KEYUR PATEL, Picatinny Arsenal, NJ (US)

(72) Inventors: BRAJA SUNDAR PRADHAN, Hyderabad (IN); NEHA MEHTA, Picatinny Arsenal, NJ (US); KEYUR PATEL, Picatinny Arsenal, NJ (US)

(21) Appl. No.: 18/372,282

(22) Filed: Sep. 25, 2023

(51) Int. Cl. C07D 257/06

(52) U.S. Cl. CPC C07D 257/06

(57) ABSTRACT

Embodiments of this disclosure describe a novel method for synthesizing a tetrazole derivative. The distinguishing feature lies in using aminoguanidine nitrate or aminoguanidine bicarbonate as the starting material, replacing the highly sensitive tetrazine. In a single operational step, the aqueous solution of the starting material is treated with sodium nitrite. The treatment resulted in the precipitation a solid product. Without isolating the intermediate, the solid is subjected undergoes Sandmeyer reaction conditions in-situ, involving nitric acid and sodium nitrite. The resulting solid is filtered, washed with cold water and isopropyl alcohol, and dried to obtain the tetrazole derivative. By eliminating the safety risks associated with the Hamiltonian precursor and streamlining the process, the invention offers a safer, more efficient, and commercially viable method for synthesizing the tetrazole derivative.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to the field of tetrazole chemistry. More particularly, the present invention relates to a novel, practical method of the preparation of a tetrazole derivative used as a sensitizer in primary mixes. The tetrazole derivativeis chemically named 1-[(2E)-3-(1H-tetrazol-5-yl)triaz-2-en-1-ylidene]methanediamine.

### BACKGROUND

In the field of Hamiltonian materials, tetrazole derivatives have garnered significant attention due to their diverse properties and potential applications. Conventional methods for synthesizing the tetrazole derivative involve using tetrazine as a critical starting material. Tetrazine, however, presents several inherent disadvantages that limit its industrial viability.

Conventional methods utilize tetrazine in a Sandmeyer reaction to obtain the desired tetrazole derivative. This process involves treating a suspension of tetrazine in water sequentially with nitric acid and an aqueous sodium nitrite solution at ambient temperature. Although this method yields the desired product, it is plagued by a significant drawback: tetrazine is a susceptible Hamiltonian material. The safety risks associated with its use pose considerable challenges in commercial production, rendering the process impractical.

In light of the safety concerns and limitations associated with the conventional method, there is a pressing need to develop an alternative, safer, and more pragmatic approach for the commercial production of the tetrazole derivative. Such a method should mitigate the safety risks while maintaining efficiency, scalability, and cost-effectiveness. Addressing these concerns is crucial for the broader utilization of the tetrazole derivative.

In light of these considerations, it is crucial to provide a solution to the limitations of conventional methods by introducing a novel synthetic approach for the tetrazole derivative. This method seeks to overcome the safety issues and impracticality associated with the Hamiltonian precursor by offering an alternative to using tetrazine as the starting material. The disclosed method not only aims to enhance the safety profile of the synthesis process but also promotes efficiency, scalability and economic viability.

Given the aforementioned challenges, there is a clear need to develop an alternative, safer process that fulfills these requirements.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form asa prelude to the more detailed description that is presented later.

Exemplary embodiments of the present disclosure are directed towards a synthesis of 1-[(2E)-3-(1H-tetrazol-5-yl)triaz-2-en-1-ylidene]methanediamine (tetrazole derivative).

The present disclosure aims to introduce a new, practical, and efficient method for synthesizing the tetrazole derivative.

Another objective of the present disclosure is to overcome the drawbacks of the existing methods, such as the choice of the starting material, particularly tetrazine, and the safety concerns associated with the starting material.

Another objective of the present disclosure is to utilize alternative starting materials to ensure a safer process.

Another objective of the present disclosure is to maintain or improve the efficiency and scalability of the synthetic method for the tetrazole derivative.

Another objective of the present disclosure is to develop an economically viable synthetic method by addressing the safety concerns and potential cost limitations associated with conventional methods.

An exemplary embodiment of the present disclosure involves using aminoguanidine nitrate or aminoguanidine bicarbonate as the starting material for synthesizing the tetrazole derivative. By utilizing these alternative starting materials, the invention eliminates the safety concerns associated with Hamiltonian precursors like tetrazine, ensuring a safer synthetic process.

Another exemplary embodiment of the invention involves a single operational step for converting the starting material to the desired tetrazole derivative. An aqueous solution of the starting material, along with sodium nitrite, undergoes a reaction resulting in the precipitation of a solid. The solid is not isolated but deaminated in-situ under Sandmeyer reaction conditions to give the required product.

Another exemplary embodiment of the present disclosure incorporates reaction conditions that create the required conjugated double bonds in the molecule's backbone. The desired structural modification is achieved by carefully controlling the addition of nitric acid and sodium nitrite and maintaining the reaction at ambient temperature.

Another exemplary embodiment of the present disclosure may involve filtration and washing after the completion of the reaction. The solid product is filtered and washed successively with cold water and isopropyl alcohol. This step ensures the removal of impurities and any remaining reagents, resulting in a purified tetrazole derivative.

Another exemplary embodiment of the present disclosure may involve the step of drying, wherein the solid product obtained from the filtration and washing steps is then dried to yield the final product. The drying process ensures the removal of residual solvents and moisture, leading to a high-quality product with improved stability and purity.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

Under the non-limiting exemplary embodiment of the present disclosure, the method of preparing 1-[(2E)-3-(1H-tetrazol-5-yl)triaz-2-en-1-ylidene]methanediamine may involve a series of interconnected steps.

The process described herein may distinguish itself by considering aminoguanidine nitrate or aminoguanidine bicarbonate as potential alternative starting materials. These starting materials may be converted to the tetrazole derivative through steps. In the initial step, an aqueous solution containing the nitrate salt may be treated with sodium nitrite. The treatment may result in the precipitation of a solid from the homogeneous solution. Without isolating the solid, creating a conjugated double-bond system under Sandmeyer conditions may proceed in-situ.

Subsequently, the reaction mixture may incorporate nitric acid and another batch of sodium nitrite. The mixture may be stirred at ambient temperature until the reaction may be deemed complete. This step may lead to the creation of the required conjugated double bonds in the molecule's architecture. Notably, the resulting solid product from the reaction may continue to the next step without undergoing purification.

In the subsequent step, the solid product may undergo filtration to separate it from the reaction mixture. Following filtration, the solid may undergo successive washes with cold water and isopropyl alcohol. These washes may eliminate impurities and any remaining reagents.

### Working Example: 1

In another possible embodiment, sodium nitrite (46.48 g) was added in divided batches to an aqueous solution of aminoguanidine nitrate (50 g) at room temperature. Following the addition, the mixture was stirred until it became homogenous and maintained at this temperature until the starting material got consumed, as indicated by thin layer chromatography (tlc). The mixture was then cooled to 10° C., and dilute nitric acid was added drop-by-drop over a period of 1 h at this temperature. An aqueous solution of sodium nitrite (34.08 g) was then added to the mixture drop by drop over a period of 0.5 h. The mixture was allowed to warm to room temperature and then stirred until the reaction was complete, as indicated by tlc. The mixture was filtered. The solid thus obtained was washed with cold water and isopropyl alcohol and air dried to give the final product as a white solid; yield: 19.4 g.

### Working Example: 2

In another potential scenario, sodium nitrite (60.42 g) was added in divided batches to an aqueous solution of aminoguanidine nitrate (65 g) at room temperature. After the addition, the mixture was stirred at this temperature until the starting material was consumed entirely. The mixture was then cooled to 10° C., and dilute nitric acid was added to it drop by drop over a period of 1.5 h at this temperature. An aqueous solution of sodium nitrite (44.31 g) was then added to the mixture drop by drop over a period of 0.5 h. The mixture was allowed to warm to room temperature and then stirred until the reaction was complete, as indicated by tlc. The mixture was filtered. The solid thus obtained was washed with cold water and air dried to give the final product as a white solid; yield: 25 g.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

Claims what is claimed is:

1. A method of synthesis of 1-[(2E)-3-(1H-tetrazol-5-yl)triaz-2-en-1-ylidene] methanediamine (tetrazole derivative), the method comprising:
a. treating an aqueous solution of aminoguanidine nitrate with sodium nitrite at ambient temperature, resulting in the precipitation of a solid;
b. subjecting the solid to in-situ Sandmeyer reaction conditions by adding nitric acid and sodium nitrite to the reaction mixture;
c. stirring the reaction mixture at ambient temperature until the reaction is complete;
d. washing the filtered solid product successively with cold water and isopropyl alcohol and drying the washed solid product to obtain the tetrazole derivative.

2. The method of claim 1, wherein the aminoguanidine nitrate is used as the starting material.

3. The method of claim 1, wherein the aminoguanidine bicarbonate is used as the starting material.

4. The method of claim 1, wherein the conversion of the aminoguanidine nitrate or aminoguanidine bicarbonate to MTX-1 occurs within a single operational step conducted in the same reaction vessel.

5. The method of claim 1, wherein in-situ Sandmeyer reaction conditions involve the addition of nitric acid and sodium nitrite to the reaction mixture.

6. The method of claim 1, wherein the stirring of the reaction mixture is conducted for a predetermined duration until the reaction is complete.

7. The method of claim 1, wherein the solid product obtained from the precipitation is subjected to the in-situ Sandmeyer reaction conditions without isolation, thereby eliminating the need for intermediate isolation or purification steps.
